(19) United States
(12) Patent Application Publication
(10) Pub. No.: US 2002/0160457 A1
(43) Pub. Date: Oct. 31, 2002

(54) Rapid diagnostic test to identify animals vaccinated with brucella abortus RB 51

(76) Inventor: Gerhardt Schurig, Blacksburg, VA (US)

Correspondence Address: THE TECHNOLOGY LAW OFFICES OF VIRGINIA, P.O. Box 818, Middleburg, VA, 20118, US

(21) Appl. No.: 10/098,793

(22) Filed: Mar. 14, 2002

Related U.S. Application Data
(60) Provisional application No. 60/286,281, filed on Apr. 25, 2001.

(51) Int. Cl. C12Q 1/68; C12N 15/09

(52) U.S. Cl. 435/69.3

(57) ABSTRACT

This invention relates to a kit for rapid detection of whether animals, particularly cattle, have been vaccinated with B. abortus RB 51. This vaccination is a brucellosis inhibitor in cattle and the kit includes latex beads which are used, in the latex agglutination test or enzyme-liked immunosorbent assay, to determine whether an animal has, in fact, been vaccinated.

This invention is based upon and relies upon and incorporates the subject matter of an earlier filed provisional application Serial No. 60/286,281, filed Apr. 25, 2001, by the same inventor, Gerhardt Schurig, and upon which priority is based.

This invention relates to a diagnostic method capable of detecting animals vaccinated with Brucella abortus RB 51. Specifically, the present invention provides methods for the preparation of appropriate antigens from B. abortus RB 51, and procedures to carry out a rapid, on-the-site test to identify the B. abortus RB 51 vaccinated animals.

### BACKGROUND

B. abortus is a bacterium that causes brucellosis in animals and humans. See “Zoonoses and communicable diseases common to man and animals”, by P. Acha et al, 1980, Pan American Health Organization, pp. 28-45, which is hereby incorporated by reference herein. This bacterium is the primary causative agent of brucellosis in cattle. The disease results in abortions and infertility in the affected cattle herds, leading to devasting economic losses to farmers as well as beef and dairy industries. Specific cell-mediated, but not, antibody, immune responses are crucial for protecting animals against brucellosis. B. abortus vaccine strain RB51 confers protection by inducing such cell-mediated immune responses. See “Biological properties of RB 51: a stable rough strain of Brucella abortus, by G. Schurig et al, 1991, Veterinary Microbiology, 28: 171-188, and “Overexpression of protective antigen as a novel approach to enhance vaccine efficacy of Brucella abortus strain RB 51” by Vemulapalli et al, 2000, Infectious Immunity, 68: 3286-3289, both of which are hereby incorporated by reference herein.

B. abortus strain is currently employed as the official vaccine for cattle brucellosis in the U.S.A. and several other countries around the world. A major advantage of using this vaccine strain is that the vaccinated animals, unlike the infected ones, do not produce antibodies to a specific antigen, O antigen, which is present in abundant quantities in virulent strains as mentioned in the 1991 reference by Schurig, above. This feature of B. abortus vaccine strain RB 51 enables the utilization of O antigen-based serological tests for the differentiation of infected and non-infected or vaccinated animals, which is critical for the success of the disease eradication programs. Until now, there has been no rapid detection test available for differentiating between B. abortus strain RB 51-vaccinated and normal [non-vaccinated and not infected] animals. Such a test greatly helps in the efficient implementation of B. abortus strain RB 51 vaccination and brucellosis eradication programs. Since B. abortus strain RB 51 preferentially induces cell-mediated immune responses, conventional anti-body-based tests are not suitable for rapidly identifying the vaccinated animals. All the presently tests using the extracted antigen preparations. Using the low molecular weight antigen preparations described in this invention, rapid, qualitative or semi-qualitative antibody-based assays developed for on-the-site identification of B. abortus strain RB 51 vaccinated animals. This part of the invention relies on routine techniques in the field of immunodiagnostics, well known to those of ordinary skill in the art.

### DETAILED DESCRIPTION OF THE INVENTION

B. abortus strain RB 51 is grown in Tryptic soy broth (TSB) or on Tryptic soy agar (TSA) plates. Chloramphenicol at the concentration of 30 μg/ml is added to the broth or agar while culturing bacteria containing the broad-host-range plasmid pBBR1MCS or its derivatives.

Overexpression of Selected B. abortus Antigens in Strain RB 51.

The genes encoding low molecular weight (less than 30 kildaltons in size ) proteins of B. abortus are obtained from the genomic DNA of B. abortus via polymerase chain reaction or conventional gene cloning methods. Gene sequence information for proteins with the described characteristics are obtained from either the databases or published literature. These genes along with or without their promoters are cloned into broad host range plasmids such as pBBR1MCS or its derivatives. The resulting recombinant plasmids are electroporated into B. abortus strain RB 51, and the recombinant colonies are isolated on a TSA plate containing chloramphenicol or other appropriate antibiotic. Overexpression of the cloned gene product is confirmed by electrophoresis as described available tests can only be performed in a properly equipped laboratory by appropriately trained personnel. Additionally, the presently available antigen reagents cause the development of high backgrounds, forcing the inclusion of several kinds of control tests and appropriate analysis of the results before judging whether a sample is positive for B. abortus RB 51 vaccination. The present invention describes methods for developing a rapid detection test that provides clear and unambiguous results and can be performed on the farm or on the site by even trained personnel.

### SUMMARY OF THE INVENTION

The present invention relates to diagnostic methods capable of detecting animals vaccinated with Brucella abortus RB 51. Specifically, the invention relates to preparation of antigens from B. abortus strain RB 51 which are suitable for use in developing rapid diagnostic tests for identifying B. abortus strain RB 51 vaccinated animals. Various methods to extract low molecular weight anitigens that show high levels of specificity for Brucella species are described. These methods include (1) precipitation of low molecular weight antigens present in the late stage cultures of B. abortus strain RB 51; (2) lysis of strain RB 51 and separation of low molecular weight antigens; (3) overexpression and extraction of selected proteins of B. abortus in strain RB 51 using multicopy plasmids which based on pBBR1MCS, its derivatives or unrelated plasmids.

This invention further relates to the development of rapid, serological diagnostic in the literature.

Extraction of Low Molecular Weight Antigens from B. abortus Strain RB 51 or Its Recombinant Versions.

B. abortus RB 51 or its recombinant strains are grown in TSB or other suitable medium to their late log or early stationary stage. The bacteria are then harvested by centrifugation of the cultures at 8,000×g for 10 minutes. The harvested bacteria are resuspended in 1/50 volume (of the original culture volume ) of 1% (wt/vol) sodium dodecyl sulfate solution. The suspensions are rocked at room temperature for 3 hours to facilitate the lysis of the bacteria followed by ultrasonication for 5 minutes. Subsequently, the lysates are centrifuged at 15,000× g for 30 minutes, and the clear supernatants are transferred to clean containers. The final solution is then passed through a filter containing pores that allow the passage of antigens of 30 or less kilodaltons in size. The solution containing the low molecular weight antigens is concentrated by freeze-drying and stored at room temperature until use in the development of rapid detection assay.

Preparation of Low Molecular Weight Antigen from the Culture Supernatants.

B. abortus RB 51 or its recombinant strains are grown in TSB or other suitable medium to their late stationary stage. The bacteria are then harvested by centrifugation of the cultures at 10,000× g for 15 minutes. The culture supernatants are transferred to clean glass flasks. To this solution, trichloro-acetic acid (TCA) is added to a final concentration of 20% (wt/vol). The solution is constantly stirred with a magnetic stirrer while adding TCA. The mixture is centrifuged at 15,000× g for 30 minutes, and the pellets resuspended in 1/1000 volume of the original culture volume of 1M Tris solution. Care should be taken in handling the mixtures containing TCA, and it should be disposed according to the established chemical safety and hygiene plan. The resuspension solution is passed through a filter containing pores that allow the passage of antigens of 30 or less kilodaltons in size. The solution containing the low molecular weight antigens is concentrated by freeze-drying and stored at room temperature until use in the development of rapid detection assay.

Development of Rapid Immunodiagnostic Assays.

Using the low molecular weight antigen preparations described in this invention, diagnostic tests such as latex agglutination test, dip-stick/dot-blot immunoassay, or enzyme-liked immunosorbent assay can be developed for rapid detection of B. abortus RB 51 vaccinated animals. For a review of immunological and immunoassay procedures in general, see Basic and Clinical Immunology, 7^{th} Ed. (D. Stites and A. Terr, 1991). Immobilizing the prepared low molecular weight antigens on to latex beads, nitrocellulose or nylon membrane, or polystyrene micro tubes is the first step in developing several configurations of the diagnostic test. Diagnostic kits are prepared using the immobilized antigen matrices along with the appropriate common reagents and positive and negative control serum samples. To perform this test, serum or plasma from an animal is collected and diluted in a buffer solution and mixed with the immobilized antigen matrix. After incubating for several minutes (typically 10 to 15 minutes ) the presence of specific antibody in the sample serum can be detected by clumping of the beads in case latex agglutination test. In case of the other two assay formats, additional reagents (secondary antibody conjugated to an enzyme and substrate for the enzyme) added in sequence and the development of a colored spot (dip-stick/dot-blot immunoassay ) or colored solution (enzyme-liked immunosorbent assay) indicates the presence of antibodies to B. abortus RB 51.

Having described several preferred embodiments it will be obvious to those of ordinary skill in the art to make changes and additions without departing from the scope of the appended claims.

1. A diagnostic test method for detecting whether animals have been vaccinated with Brucella abortus RB 51 comprising:
cloning gene sequence information for proteins into broad host range plasmids,
electroporating the resulting recombinant plasmids in B. abortus strain RB 51, isolating the recombinant colonies on a TSA plate, and
confirming overexpression of the cloned gene product by electrophoresis.

2. The method of claim 1 in which said cloning step includes cloning the promoters of said genes with said genes.

3. The method of claim 1 wherein said TSA plate has chloramphenicol or another appropriate antibiotic thereon.

4. The method of claim 1 wherein said gene sequence information is obtained from databases.

5. The diagnostic test method for detecting whether animals have been vaccinated with Brucella abortus RB 51, said method comprising combining a sample of said animal with a low molecular weight antigen from B. abortus strain RB 51.

6. A method as in claim 5 wherein said low molecular weight antigen is produced as follows:
growing B. abortus 51 in a suitable medium to their late log or early stationary stage,
harvesting the bacteria by centrifugation,
placing said bacteria in a solution and rocking at room temperature and ultrasonification,
filtering said solution to allow passage of only antigens of 30 or less kilodaltons,
freeze drying said low molecular antigens until use.

7. A method of detecting whether animals have been vaccinated with Brucella Abortus RB 51, said method comprising
immobilizing low molecular weight antigens onto beads,
preparing diagnostic kits using said immobilized antigen matrices with appropriate reagents and positive and negative control samples,
collecting serum or plasma from an animal and collecting and diluting in a buffer solution and then mixing with the immobilized antigen matrix,
detecting the presence of Brucella abortus RB 51 by clumping of the beads in the test.

8. A diagnostic kit for detecting whether an animal has been vaccinated with Brucella abortus RB 51, said kit comprising
low molecular weight antigens in an immobilized antigen matrix, said matrix adapted to be mixed with serum or plasma from said animal to detect the presence of Brucella abortus RB 51.
